(19) United States
(12) Patent Application Publication
(10) Pub. No.: US 2020/0230276 A1
(43) Pub. Date: Jul. 23, 2020

(54) DEVICE AND METHOD FOR IN-SITU PRODUCTION OF A STERILISATION GAS AND STERILISATION OF OBJECTS AND THEIR USE

(71) Applicant: Fraunhofer-Gesellschaft zur Förderung der angewandten Forschung e.V., Munich (DE)

(72) Inventors: Gunther Kolb, Mainz (DE); Stefan Neuberg, Mainz (DE); Helmut Pennemann, Mainz (DE); Richard Thiele, Mainz (DE); Peter Wernig, Mainz (DE); Martin Wichert, Mainz (DE)

(73) Assignee: Fraunhofer-Gesellschaft zur Förderung der angewandten Forschung e.V., Munich (DE)

(21) Appl. No.: 16/632,181

(22) PCT Filed: Jul. 19, 2018
(86) PCT No.: PCT/EP2018/069586

§ 371 Date: Jan. 17, 2020

(30) Foreign Application Priority Data
Jul. 19, 2017 (DE) 10 2017 212 337.0

(51) Int. Cl. A61L 2/20; C07D 301/22

(52) U.S. Cl. CPC A61L 2/206; C07D 301/22; A61L 2202/25; A61L 2202/13; A61L 2202/24; A61L 2202/11

(57) ABSTRACT

An apparatus and a method are provided for the on-site production of a sterilizing gas and sterilization of objects. In a first step of the method, an alcohol is catalytically converted to an alkene oxide. The alkene oxide is subsequently mixed with an inert gas comprising water vapor and nitrogen to produce a sterilizing gas. Finally, the objects are sterilized with the sterilizing gas. The only starting materials used throughout the entire method are at least one alcohol and a gas comprising oxygen and nitrogen. An apparatus is provided in which the method can be performed.

### CROSS-REFERENCE TO RELATED APPLICATIONS

This application is a 371 nationalization of international patent application PCT/EP2018/069586 filed Jul. 19, 2018, which claims priority under 35 USC § 119 to Germany patent application DE 10 2017 212 337.0 filed Jul. 19, 2017. The entire contents of each of the above-identified applications are hereby incorporated by reference.

### BRIEF DESCRIPTION OF THE DRAWINGS

FIG. 1 shows a section of the flow diagram for the reaction module of an apparatus according to the invention; and

FIG. 2 illustrates the flow diagram of the inert gas generator in an apparatus according to the invention.

### DETAILED DESCRIPTION

The present invention relates to an apparatus and a method for the on-site production of a sterilizing gas and sterilization of objects. In a first step of the method, an alcohol is catalytically converted to an alkene oxide. The alkene oxide is subsequently mixed with an inert comprising water vapor and nitrogen gas to produce a sterilizing gas. Finally, the objects are sterilized with the sterilizing gas. The only starting materials used throughout the entire method are at least one alcohol and a gas comprising oxygen and nitrogen. The present invention additionally also relates to an apparatus in which the method can be performed.

An object is completely freed of germs during the sterilization. This is usually done by killing all of the pathogens adhering to the object.

Various procedures can be selected here depending on the nature of the object to be sterilized. If the object is heat-stable, it may be sterilized using dry hot air at temperatures of about 180° C. Somewhat lower temperatures are used for autoclaving. Here, the contaminated object is disinfected under elevated pressure at about 120° C. Heat-sensitive objects, for example objects made from plastic, are sterilized in contrast using chemical substances (epox-ides, formaldehyde) or radiation (UV, gamma or x-ray radiation).

The epoxide ethylene oxide (EO) is a particularly popular sterilizing gas for temperature-sensitive medical products. It acts reliably against viruses, fungi and bacteria and a substitution of this gas is unimaginable in the medical sec-tor.

A disadvantage with the use of EO, however, is that it is toxic and readily in-flammable and can form highly explosive mixtures with air (the lower explosive limit is 2.6% and the upper explosive limit is 100%). EO sterilization can therefore only be effected under safety constraints and must be performed by trained specialized personnel. In addition, EO is harmful to the environment and may only be released into the environment in low concentrations. These factors, and also the transport and the storage of the gas, make sterilization with EO, but also sterilization with other alkene oxides, expensive and unat-tractive as on-site methods.

Many medical facilities therefore outsource the sterilization process. They send their contaminated medical products to external contractors and have them sterilized there. This approach is unsatisfactory for a number of reasons.

Firstly, EO is used in undiluted form for sterilization at the external contractors (e.g. Sterigenics, Wiesbaden). There are 2 kg of EO for a pallet loaded with items to be sterilized. This increases the desorption time and makes the sterilization method inefficient and time-consuming, since EO first has to be de-sorbed completely from the surface of the sterilized objects before the sterilized objects are able to be used again. Secondly, the items to be sterilized have to be transported over great distances to the centrally located facilities of the external contractor and returned again to the end consumers.

Proceeding from this, it was therefore the object of the present invention to specify a method and a corresponding apparatus with which the chemical sterilization of temperature-sensitive objects can be performed rapidly, safely and directly on site in the hospitals, doctor's offices and laboratories.

The invention provides a method for the on-site production of a sterilizing gas and subsequent sterilization of objects, comprising the following method steps:
a) catalytic conversion of an alcohol to an alkene oxide,
b) production of the sterilizing gas by mixing the alkene oxide with an inert gas comprising water vapor and nitrogen and
c) sterilization of the objects with the sterilizing gas,

wherein the only starting materials used for the method are at least one alcohol and a gas comprising oxygen and nitrogen.

This method is preferably performed continuously.

The method according to the invention is superior to the sterilization methods known to date from the prior art, since the starting materials used here are not explosive and are readily available. Sterilization of contaminated objects can then be performed without problems in a decentralized manner (on-site).

In addition, the alkene oxide which is critical in the handling is only produced in situ from an alcohol, with the catalytic conversion advantageously comprising a catalytic dehydration of the alcohol to give the alkene in a first stage and an epoxidation of the alkene in the presence of a catalyst in a second stage. The safety measures normally employed for the storage of alkene oxides are dispensed with by the in-situ production. At the same time, the safety risk is also reduced in comparison to conventional EO sterilization methods.

The dilution of the alkene oxide in the method according to the invention in step b) also has positive effects. Since the alkene oxide is mixed with a gas comprising water vapor and nitrogen prior to the sterilization of the object and is not used in undiluted form, any desired concentration can be set. The variable composition of the sterilizing gas makes more universal use of the method possible. Furthermore, a lower concentration of the alkene oxide in the sterilizing gas also automatically leads to a shorter desorption time after sterilization has been performed and hence to a higher economic efficiency of the entire method. The addition of an inert gas comprising water vapor and nitrogen additionally increases the moisture content of the sterilizing gas and as a result makes the pathogens more sensitive to the alkene oxide. The sterilizing effect is thus enhanced.

In terms of sustainability, the method according to the invention can also be carried out with bioalcohol and using green electricity. A completely CO₂-neutral method would be achieved as a result.

In a preferred embodiment, the sterilizing gas after method step c) is disposed of on-site in an additional method step d), preferably by adding a portion of the gas comprising oxygen and nitrogen and completely catalytically oxidizing the alkene oxide in the presence thereof, particularly preferably at temperatures of 150 to 300° C. The catalyst used is preferably a platinum-containing catalyst, for example having a porous support material made of aluminum oxide. Particularly preferably, the catalyst has an operational stability of more than 35 000 hours. In this way, frequent replacement and frequent interrup-tions to the method for maintenance purposes can be avoided.

The complete oxidation of the alkene oxide can ensure that the required envi-ronmental standards are complied with. The alkene oxide is converted to a mixture of the non-toxic substances nitrogen, carbon dioxide and water vapor. As a result, the process offgases contain less than 1 ppm of alkene oxide. Only traces of alkene oxide are thus released into the environment and the method can be performed in medical facilities, for example hospitals or doctor's practices. In addition, heat, which by way of heat integration can be used at another point in the method, is released when disposing of the sterilizing gas, since the complete catalytic oxidation of the alkene oxide in method step d) proceeds in a highly exothermic manner.

In a further preferred embodiment, the alcohol is selected from the group consisting of ethanol, propanol, butanol and mixtures thereof, wherein particular preference is given to using ethanol.

Ethanol has the advantage that—in contrast to EO—it is available anywhere in the world, which increases exportability and hence also the competitiveness of the method.

If the alcohol is ethanol, a gas mixture composed of ethene and water vapor may be generated in the first stage of the conversion from the alcohol to the alkene oxide. Depending on the catalyst, the reaction temperature and the reactor loading, the following slightly endothermic/slightly exothermic reactions (1) and (2) having different product selectivities may take place in parallel:

$$\begin{matrix}{C}_{2} {H}_{5} OH \to {C}_{2} {H}_{4} + {H}_{2} O {Δ}_{R} {H}^{0} = + 45.8 \frac{ kJ}{mol} & ( 1 ) \\ 2 {C}_{2} {H}_{5} OH \to {H}_{5} {C}_{2} - O - {C}_{2} {H}_{5} {Δ}_{R} {H}^{0} = - 23.8 \frac{kJ}{mol} & ( 2 )\end{matrix}$$

Low reaction temperatures favor intermolecular dehydration and result in the formation of diethyl ether (2), whereas high reaction temperatures promote the formation of ethene by way of intramolecular dehydration (1). The ethanol conversion increases with increasing temperature. Under isothermal conditions and at higher temperatures, an increase in the reactor load may result in a slight decrease in the ethanol conversion and the ethene selectivity and a slight increase in the diethyl ether selectivity. Preferred catalyst loads or space velocities, which are defined as the ratio of reactant volume flow rate to the catalyst mass, are from 8 to 12 l/(h·g_{cat}).

In the second stage, if the alcohol selected was ethanol, ethene can be reacted with the gas comprising oxygen and nitrogen to give a gas mixture composed of EO, carbon dioxide, water vapor and nitrogen. Depending on the reaction conditions, the catalyst used and inhibitors added, various exothermic reactions as per the equations (3) to (5) may take place:

$$\begin{matrix}{C}_{2} {H}_{4} + 1 / 2 {O}_{2} \to {C}_{2} {H}_{4} O {Δ}_{R} {H}^{0} = - 105.18 \frac{ kJ}{mol} & ( 3 ) \\ {C}_{2} {H}_{4} + 3 {O}_{2} \to 2 {CO}_{2} + 2 {H}_{2} O {Δ}_{R} {H}^{0} = - 1324.0 \frac{kJ}{mol} & ( 4 ) \\ {C}_{2} {H}_{4} O + 5 / 2 {O}_{2} \to 2 {CO}_{2} + 2 {H}_{2} O {Δ}_{R} {H}^{0} = - 1218.9 \frac{kJ}{mol} & ( 5 )\end{matrix}$$

In this case, reactions (3) and (4) are parallel reactions, while the total oxidation of EO (5) may occur as a subsequent reaction, though this is not observed in practice for kinetic reasons.

It is further preferred if in the second stage of the conversion of the alcohol to the alkene oxide the catalyst used is an epoxidation catalyst, optionally coupled with a dehydration catalyst in the first stage of the conversion of the alcohol to the alkene oxide, wherein the epoxidation catalyst is in particular a copper oxide/silver-based or a silver-based catalyst and the dehydration catalyst is selected in particular from the group consisting of TiO₂/γ-Al₂O₃—, La-P-HZSM-5-, HZSM-5-zeolite-, Ag₃PW₁₂O₄₀—, H₃PW₁₂O₄₀-MCM-41-, W-silicate-heteropolyacid- and MgO—Al₂O₃/SiO₂-based catalysts and also mixtures thereof.

If the alcohol in the method according to the invention is ethanol, the epoxidation catalyst used is advantageously a catalyst consisting of silver and copper oxide. A high EO yield of up to 12% is achieved with this catalyst.

Preferred operating temperatures for the dehydration catalyst are between 220 and 500° C., in particular between 440 and 460° C. The epoxidation catalyst is advantageously used at temperatures in the range from 250° C. to 350° C. for the catalysis of the epoxidation reaction.

One difficulty in the epoxidation of the alkene to the alkene oxide may reside in a competing parallel reaction, specifically in the complete oxidation of the alkene to carbon dioxide and water. In this case it is advantageous for cesium to be added to the epoxidation catalyst. This can improve the long-term stability and selectivity of the catalyst. The addition of chloroethane or vinyl chlo-ride to the alkene can also suppress total oxidation.

In a preferred embodiment of the method, the gas comprising oxygen and nitrogen is ambient air.

The inert gas comprising water vapor and nitrogen is obtained in situ from the alcohol and the gas comprising oxygen and nitrogen. The reaction to give the inert gas comprising water vapor and nitrogen preferably takes place over a platinum-based catalyst which optionally contains a promoter selected from the assortment consisting of cerium, molybdenum, manganese and mixtures thereof and particularly preferably is supported on a porous γ-aluminum oxide layer. The reaction preferably proceeds here continuously and in parallel with the conversion of alcohol to alkene oxide. It is additionally advantageous for a portion of the water vapor to be condensed back out of and separated off from the freshly produced inert gas comprising water vapor and nitrogen by cooling down to temperatures below 90° C., particular preferably below 60° C.

If the alcohol is ethanol, the inert gas comprising water vapor and nitrogen predominantly consists of nitrogen and water vapor, but it also contains relatively small proportions of carbon dioxide and methane and also traces of other compounds (less than 1 mol %). The reaction of ethanol and ambient air as the gas comprising oxygen and nitrogen proceeds in a highly exothermic manner as per the following reaction equation (6):

$${C}_{2} {H}_{5} OH + 3 {O}_{2} + 11.2857 {N}_{2} \to 2 {CO}_{2} + 3 {H}_{2} O + 11.2857 {N}_{2} \\ {Δ}_{R} {H}^{0} = - 1278.1 \frac{kJ}{mol} \qquad ( 6 )$$

This can lead to a volume increase which is more than 5%, preferably 6.5%.

The inert gas comprising water vapor and nitrogen preferably has a relative humidity of 40% to 70%, very particularly preferably of 55% to 65%. This is equivalent to a proportion of water vapor in the inert gas comprising water vapor and nitrogen of preferably 3 mol % to 20 mol %, in particular 6 mol % to 10 mol %.

The inert gas comprising water vapor and nitrogen is preferably generated at elevated temperature. At a temperature above 290° C., the alcohol is completely converted and the yield of inert gas is increased. In addition, the inert gas generated is sterile on account of the heat.

In a further preferred embodiment of the method, the sterilizing gas contains 3 mol % to 25 mol %, preferably 5 mol % to 22 mol %, particularly preferably 6 mol % to 10 mol %, in particular 6 mol % to 8 mol %, of the alkene oxide.

The objects are preferably placed into a sterilizing chamber in order particularly preferably to firstly precondition them there with the inert gas comprising water vapor and nitrogen. The preconditioning is advantageously performed here at temperatures between 40 and 80° C. It is particularly preferred for the objects to be sterilized with the sterilizing gas in method step c) for a duration from 0.5 to 300 minutes, in particular at temperatures of 40 to 80° C., for example of 55 to 65° C.

Preconditioning can be achieved by flooding the sterilizing chamber once or flushing it multiple times with the inert gas comprising water vapor- and nitrogen. The objects to be sterilized are moistened by the preconditioning, with the result that adhering viruses, fungi and bacteria are capable of absorbing the alkene oxide since they swell as a result of the moisture on the cell surface.

The sterilizing chamber after method step c) is preferably flushed with the inert gas comprising water vapor and nitrogen and/or is evacuated using a vacuum pump in order to completely free the objects of the alkene oxide.

The inert gas comprising water vapor and nitrogen is particularly suitable for this flushing procedure, since the nitrogen present at a high proportion diffus-es more rapidly and thus can accelerate desorption. In contrast, a gas predominantly containing carbon dioxide would not be suitable for flushing the sterilizing chamber. Furthermore, the evacuation using the vacuum pump also accelerates the desorption process of the alkene oxide present in the sterilizing gas.

It is additionally preferable for the energy released in method step a) to be used for the heating of at least a portion of the gas comprising oxygen and nitrogen, for example for method step d).

In this way, the sterilizing gas, which comes out of the sterilizing chamber at a temperature of less than 80° C., can be heated to a temperature range from 180 to 260° C. This temperature range is preferred for the complete oxidation of the alkene oxide for disposal in method step d).

It is additionally preferable for at least one of the method steps to be performed in a micro process engineering component, particularly preferably in a microchannel reactor.

Implementation in a microchannel reactor allows even highly exothermic reactions to be carried out isothermally.

The inventive apparatus for the on-site production of a sterilizing gas and subsequent sterilization of objects comprises at least one inlet for an alcohol and at least one inlet for a gas comprising oxygen and nitrogen and also an inert gas generator, a reaction module and a sterilizing chamber, wherein both the inert gas generator and the reaction module possess direct fluidic connections to the at least one inlet for the alcohol and the at least one inlet for the gas comprising oxygen and nitrogen and are connected upstream of the sterilizing chamber.

This apparatus may be set up in a decentralized manner in doctor's practices, so that the sustainability of the sterilization process is increased since the transport of the objects to be sterilized is omitted. The on-site installation of the apparatus may additionally shorten the sterilization time and an improved time use is guaranteed for the objects to be sterilized, for example medical products. Hazardous materials deliveries of relatively large alkene oxide con-tainers are also no longer necessary and the use of bottle-stored, small quan-tities of alkene oxide, which in the future are to be heavily regulated or for-bidden by the authorities, can be avoided.

In addition, the reaction module can have at least one micro process engineering component, preferably a microchannel reactor coated with a catalyst.

In this case, the microchannel reactor preferably consists of two series-connected, catalyst-coated microchannel reactors, wherein particularly preferably the inner wall of the reaction channels of the second microchannel reactor is coated with an epoxidation catalyst and/or the inner wall of the reaction channels of the first microchannel reactor is coated with a dehydration catalyst.

It is particularly preferable here for the inert gas generator and/or the reaction module to contain a microchannel reactor or to consist of one. These micro process engineering components have such a high surface/volume ratio that complete oxidation of the alcohol for the inert gas generation and/or epoxidation to give the alkene oxide can be carried out virtually isothermally.

One variant of the apparatus further has a combustion chamber connected downstream of the sterilizing chamber and preferably also comprises at least two independently controllable valves and also optionally a mixing chamber, wherein the mixing chamber is connected downstream of the inert gas generator and the reaction module and upstream of the sterilizing chamber, and wherein a heat exchanger is particularly preferably arranged between the mixing chamber and the sterilizing chamber.

The apparatus according to the invention can further comprise a vacuum pump, which preferably has a fluidic connection to the combustion chamber, and also optionally sensors for the detection and a control unit for the regulation of flow rate, temperature and pressure.

The apparatus according to the invention optionally further also contains a Stirling cooler into which the alkene oxide-containing stream can be fed in order to remove residual oxygen that has not reacted in the conversion of alcohol to alkene oxide. Temperatures of less than −80° C. can preferably be reached with the Stirling cooler.

In a preferred embodiment of the apparatus, the sterilizing chamber has a volume of at most 100 l, particularly preferably of at most 75 l, and particularly preferably comprises a plug for connection to the domestic electrical power supply.

The apparatus according to the invention and described above and the method according to the invention find use in the safe on-site production of a sterilizing gas, preferably for use in the food or packaging industry, for sterilizing objects or rooms, for ripening plants, particularly preferably for disinfecting medical products or medical tools, in particular for freeing from pathogens such as bacteria, viruses, fungi and other microorganisms.

The present invention will be described in more detail with reference to the following figures and the following examples, without restricting the invention to the specific parameters.

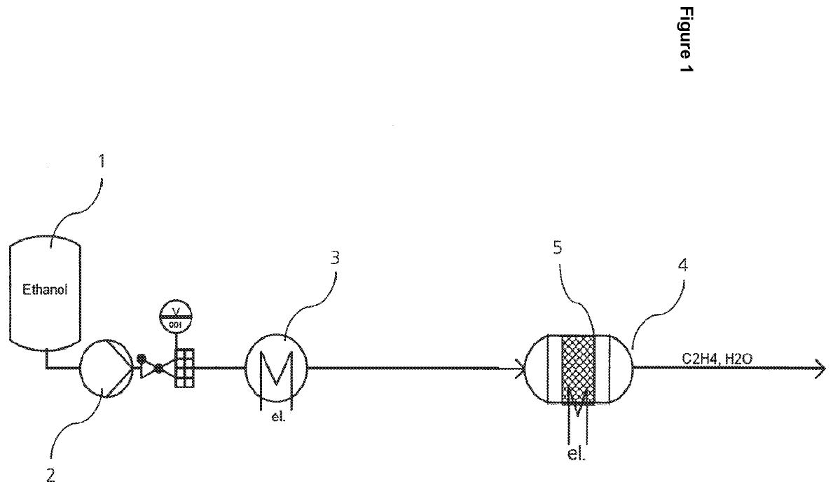

FIG. 1 shows a section of the flow diagram for the reaction module of an apparatus according to the invention. Herein, ethanol is firstly withdrawn from a storage container 1 and fed into a reactor 4 equipped with a dehydration catalyst 5 by means of a feed pump 2. Before the ethanol enters the reactor, it is brought to the desired temperature in a heat exchanger 3.

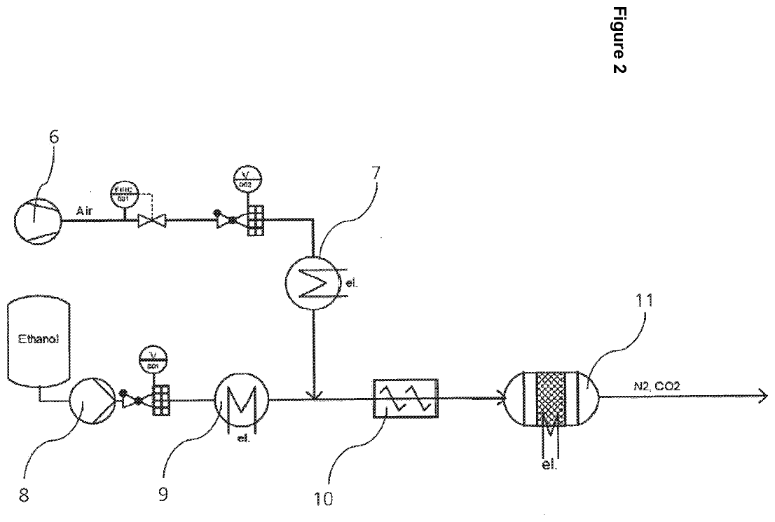

FIG. 2 illustrates the flow diagram of the inert gas generator in an apparatus according to the invention. The inert gas generator firstly draws in ambient air and compresses it by means of a compressor 6. The compressed air is then passed through a heat exchanger 7. Secondly, and in parallel with this, ethanol is passed through a second heat exchanger 9 by means of a feed pump 8.

The heated ethanol is subsequently mixed with the heated ambient air in a mixer 10, supplied to a reactor 11 equipped with a platinum-containing catalyst and converted to an inert gas comprising water vapor and nitrogen.

### Example 1

The method according to the invention was simulated using the ASPEN Plus™ software.

Conversion of Ethanol to EO

The compositions listed in table 1 below for the input stream and the product stream were found for the epoxidation of the ethene-containing intermediate.

TABLE 1
Molar composition of the input stream and the product stream in the
epoxidation stage of the reaction module for a 100% conversion of ethene.

| Component | Input stream/mol % | Product stream/mol % |
|---|---|---|
| C₂H₄ | 11.97 | 0.00 |
| O₂ | 15.97 | 0.0100 |
| N₂ | 60.09 | 62.59 |
| C₂H₄O | 0.00 | 8.31 |
| CO₂ | 0.00 | 8.31 |
| H₂O | 11.97 | 20.78 |

A decrease in volume of the product stream compared to the input stream of 4.0% and a total heat of reaction Δ_{R}H⁰ of −511.3 kJ/mol was additionally calcu-lated.

Production of the Inert Gas Comprising Water Vapor and Nitrogen

The conversion of a mixture of the gas comprising oxygen and nitrogen and ethanol (gas A) to the inert gas comprising water vapor- and nitrogen (gas B) and the optional depletion of water to form a water-depleted inert gas (gas C) were also simulated. The molar composition of these streams is listed in table 2 below.

TABLE 2
Molar composition of the input stream and output stream
of the inert gas generator.

| Component | Gas A/mol % | Gas B/mol % | Gas C/mol % |
|---|---|---|---|
| C₂H₄ | 6.54 | 0.00 | 0.00 |
| O₂ | 19.63 | 0.00 | 0.00 |
| N₂ | 73.83 | 69.30 | 78.17 |
| CO₂ | 0.00 | 12.28 | 13.85 |
| H₂O | 0.00 | 18.42 | 7.98 |

### Example 2

The method according to the invention was in addition also carried out in practice.

Conversion of Ethanol to EO

For the dehydration of ethanol to ethene in a catalyst-coated microchannel reactor, the selectivities listed in table 3 below were found at a temperature of 450° C. and a catalyst space velocity of 9.5 l/(h·g_{cat}).

TABLE 3
Overview of the selectivity of the
dehydration reaction of ethanol.

| Component | Selectivity/% |
|---|---|
| Ethene | 97.0 |
| Ethane | 0.5 |
| Propene | 0.2 |
| Butene | 2.3 |
| Methane, propane, | 0.0 |
| butane, diethyl ether | |

Production of the Inert Gas Comprising Water Vapor and Nitrogen

The conversion of ethanol and ambient air to inert gas comprising water vapor and nitrogen was carried out at three different temperatures for comparison purposes. The composition of the product streams is reported in table 4 below. The measurement error in the determination of the molar proportions was +/−2 mol %.

TABLE 4
Composition of the inert gas comprising water vapor and nitrogen
produced.

| Component | CO₂ | H₂O | C₂H₅OH | O₂ | N₂ | CO | CH₄ |
|---|---|---|---|---|---|---|---|
| Proportion @ | 9.3 | 13.6 | 0.0 | 0.0 | 76.1 | 0.1 | 0.9 |
| 350° C./mol % | | | | | | | |
| Proportion @ | 9.8 | 14.8 | 0.0 | 0.0 | 74.9 | 0.0 | 0.4 |
| 400° C./mol % | | | | | | | |
| Proportion @ | 10.2 | 15.2 | 0.0 | 0.0 | 74.3 | 0.0 | 0.3 |
| 450° C./mol % | | | | | | | |

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions herein-before or hereinafter unless expressly asserted by the Applicant to the contra-ry, to mean one or more elements selected from the group comprising A, B, . . . and N. In other words, the phrases mean any combination of one or more of the elements A, B, . . . or N including any one element alone or the one element in combination with one or more of the other elements which may also in-clude, in combination, additional elements not listed. Unless otherwise indi-cated or the context suggests otherwise, as used herein, “a” or “an” means “at least one” or “one or more.”

1. A method for the on-site production of a sterilizing gas and subsequent sterilization of objects, comprising:
performing a catalytic conversion of an alcohol to an alkene oxide;
producing the sterilizing gas by mixing the alkene oxide with an inert gas comprising water vapour and nitrogen; and
sterilizing the objects with the sterilizing gas,
wherein the only starting materials used for the method are at least one alcohol and a gas comprising oxygen and nitrogen.

2. The method as claimed in claim 1, wherein the sterilizing gas, after sterilizing the objects, is disposed of on-site by adding a portion of the gas comprising oxygen and nitrogen and completely catalytically oxidizing the alkene oxide in the presence thereof.

3. The method as claimed in claim 1, wherein the alcohol is selected from the group consisting of ethanol, propanol, butanol and mixtures thereof.

4. The method as claimed in claim 1, wherein in step a) the catalyst used is an epoxidation catalyst, optionally coupled with a dehydration catalyst, wherein the epoxidation catalyst is in particular a copper oxide/silver-based or a silver-based catalyst and the dehydration catalyst is selected in particular from the group consisting of TiO₂/γ-Al₂O₃—, La-P-HZSM-5-, HZSM-5-zeolite-, Ag₃PW₁₂O₄₀—, H₃PW₁₂O₄₀-MCM-41-, W-silicate-heteropolyacid- and MgO—Al₂O₃/SiO₂-based catalysts and also mixtures thereof.

5. The method as claimed in claim 1, that wherein the gas comprising oxygen and nitrogen is ambient air and the inert gas comprising vapour and nitrogen has a relative humidity of 40% to 70% and/or comprises water vapor in a proportion of 3 mol % to 20 mol %.

6. The method as claimed in claim 1, wherein the sterilizing gas comprises 3 mol % to 25 mol % of the alkene oxide.

7. The method as claimed in claim 1, further comprising placing the objects into a sterilizing chamber, and preconditioning the objects with the water vapor- and nitrogen-containing inert gas.

8. The method as claimed in claim 7, wherein the sterilizing chamber, after the sterilizing, is flushed with the inert gas comprising water vapor and nitrogen and/or is evacuated using a vacuum pump in order to completely free the objects of the alkene oxide.

9. The method as claimed in claim 2, wherein the energy released in the performing the catalytic conversion is used for heating of at least a portion of the gas comprising oxygen and nitrogen.

10. The method as claimed in claim 1, that wherein at least one of the method steps is performed in a micro process engineering component, and/or in a microchannel reactor.

11. An apparatus for the on-site production of a sterilizing gas and subsequent sterilization of objects, the apparatus comprising: at least one inlet for an alcohol; at least one inlet for a gas comprising oxygen and nitrogen, an inert gas generator; a reaction module; and a sterilizing chamber, wherein both the inert gas generator and the reaction module possess direct fluidic connections to the at least one inlet for the alcohol and the at least one inlet for the gas comprising oxygen and nitrogen and are connected upstream of the sterilizing chamber.

12. The apparatus as claimed in claim 11, wherein the reaction module has at least one micro process engineering component, preferably a microchannel reactor coated with a catalyst, particularly preferably consists of two series-connected, catalyst-coated microchannel reactors, wherein very particularly preferably the inner wall of the reaction channels of the second microchannel reactor is coated with an epoxidation catalyst and/or the inner wall of the reaction channels of the first microchannel reactor is coated with a dehydration catalyst.

13. The apparatus as claimed in claim 11, wherein the apparatus further has a combustion chamber connected downstream of the sterilizing chamber and preferably further comprises at least two independently controllable valves and also optionally a mixing chamber, wherein the mixing chamber is connected downstream of the inert gas generator and the reaction module and upstream of the sterilizing chamber, and wherein a heat exchanger is particularly preferably arranged between the mixing chamber and the sterilizing chamber.

14. The apparatus as claimed in claim 11, wherein the apparatus further comprises a vacuum pump, which preferably has a fluidic connection to the combustion chamber, and also optionally sensors for the detection and a control unit for the regulation of flow rate, temperature and pressure.

15. The apparatus as claimed in claim 11, wherein the sterilizing chamber preferably has a volume of at most 100 l, particularly preferably of at most 75 l, and particularly preferably comprises a plug for connection to the domestic electrical power supply.

16. The use of the apparatus as claimed in claim 11 for the safe on-site production of a sterilizing gas, preferably for use in the food or packaging industry, for sterilizing objects or rooms, for ripening plants, particularly preferably for disinfecting medical products or medical tools, in particular for freeing from pathogens such as bacteria, viruses, fungi and other microorganisms.

17. The method as claimed in claim 2, wherein the alkene oxide is completely catalytically oxidized at temperatures of 150 to 300° C.,

18. The method as claimed in claim 2, wherein the catalyst used is a platinum-containing catalyst having a porous support material made of aluminum oxide.

19. The method as claimed in claim 7, wherein the preconditioning is performed at a temperature between 40° C. and 80° C., and the sterilizing is for a duration from 0.5 to 300 minutes at a temperature in a range of 40° C. to 80° C.
